(19) United States
(12) Patent Application Publication
(10) Pub. No.: US 2012/0328550 A1
(43) Pub. Date: Dec. 27, 2012

(54) LEAVE-IN HAIR DYEING PROCESS USING A COMPOSITION COMPRISING A COLOURED POLYMER AND AN ACTIVE COSMETIC AGENT

(76) Inventors: Maxime De Boni, Shinjuku-ku (JP); Bruno Laguitton, Kremlin Bicetre (FR); Jean-Daniel Debain, Bezons (FR)

(21) Appl. No.: 12/441,208

(22) PCT Filed: Sep. 17, 2007
(86) PCT No.: PCT/FR07/51949

§ 371 Date: Nov. 2, 2009

Related U.S. Application Data
(60) Provisional application No. 60/846,753, filed on Sep. 25, 2006.
(60) Provisional application No. 60/847,120, filed on Sep. 26, 2006.
(60) Provisional application No. 60/847,391, filed on Sep. 27, 2006.

(30) Foreign Application Priority Data
Sep. 15, 2006 (FR) 0653765
Sep. 15, 2006 (FR) 0653766
Sep. 15, 2006 (FR) 0653767
Sep. 15, 2006 (FR) 0653768

(51) Int. Cl. A61K 8/92; A61K 8/89; A61K 8/90; A61Q 5/06; A61K 8/81

(52) U.S. Cl. 424/70.6

(57) ABSTRACT

The invention relates to a leave-in hair dyeing process using a composition comprising a coloured polymer and an oil. Such a process makes it possible to obtain a coloration which is easy to carry out and which preserves the cosmetic appearance of the keratin fibres.

The invention relates to a leave-in hair dyeing process using a composition comprising a colored polymer and a particular cosmetic active agent.

It is known practice to dye keratin fibers and in particular human hair with dye compositions that allow temporary dyeing to be obtained. Temporary dyeing is dyeing that shows low fastness with respect to water or to rubbing and that are removed to a large extent at the first shampoo wash. For example, patent applications EP 747 036 and EP 852 843 propose temporary colorations using water-dispersible colored polymers containing sulfo groups and comprising carbonyloxy bonds and a chromophore. U.S. Pat. No. 4,911,731 describes a temporary dyeing process that consists in applying to the hair a complex formed from a particular cationic polymer and an anionic colored polymer, and in then rinsing the hair thus dyed.

These temporary dyeings, also known as short-lasting dyeing, are generally not used for performing true dyeing: they serve essentially to heighten a faded color obtained by permanent or direct dyeing. They are not readily visible on dark hair.

The aim of the present invention is to provide a novel process for the temporary dyeing of keratin fibers that does not have the drawbacks of the colorations of the prior art, in particular a process that respects the integrity of the keratin fibers and that makes it possible to obtain strong colorations that are visible on both fair hair and dark hair, that are resistant to water or rubbing, but that are also removed to a large extent at the first shampoo wash and that are easy to use.

This aim is achieved with the present invention, one subject of which is a process for dyeing keratin fibers, comprising the application of a composition containing at least one anionic colored polymer containing an anionic chromophore and at least one cosmetic active agent chosen from a non-silicone oil, an alkylpolyglucoside surfactant, a non-associative and nonionic, amphoteric, cationic or anionic carboxylic fixing polymer, and an organosilicon compound comprising less than 6 silicon atoms, this application not being followed by a step of rinsing the fibers.

The process of the present invention makes it possible in particular to obtain rapid temporary dyeing of keratin fibers that can be performed in a single step, without damaging the keratin fibers, with a silky feel and high sheen.

The resulting coloration is instantaneously visible. The drying, for example with a hairdryer or a hood, may be performed immediately. The hair may also be dried in the open air.

The colorations obtained are short-lasting since they fade out from the very first shampoo wash and disappear within a few shampoo washes.

The process of the invention makes it especially possible to obtain a visible coloration on natural or bleached fair hair and also on dark hair that is natural or that has been dyed, for example via direct or oxidation dyeing.

The term “coloured polymer” means a polymer chain containing pendent grafts constituted of coloring units. This colored polymer may be charged. The nature of the charge of the polymer may be identical to or different than that of the colored grafts. There may be several charges per repeating unit. The overall nature of the charge in solution is pH-dependent. This polymer chain may be nonionic, anionic, cationic or amphoteric and may be totally or partially soluble in water. The overall charge of the polymer should be anionic.

Such a polymer comprises, for example, at least one unit represented by:

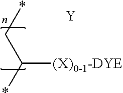

in which DYE represents the chromophore that is attached to the polymer backbone either directly or via a saturated or unsaturated, linear or branched hydrocarbon-based chain X containing from 1 to 10 carbon atoms and preferably from 1 to 6 carbon atoms, at least one of the carbon atoms of which may be replaced with a heteroatom chosen from sulfur, silicon, phosphorus, selenium, nitrogen and oxygen or an SO₂ group, the hydrocarbon-based chain possibly being substituted or unsubstituted, the polymer comprising at least one negative charge borne by the chromophore, and n represents the number of repeating units of this type present in the polymer. Generally, n is between 1 and 1000 and preferably between 1 and 500. Y is the counterion. These polymers may be block polymers or random polymers.

Substituents on the carbon-based chain that may be mentioned include alkyl, hydroxyl, alkoxy and hydroxyalkyl radicals, halogens, amino radicals or amino radicals mono- or disubstituted with an alkyl or hydroxyalkyl radical.

The chromophores that may be mentioned include radicals derived from nitrobenzene, azo, phenothiazine, xanthene, phenanthridine, phthalocyanin and triarylmethane-based dyes, and those obtained from direct dyes containing a carbonyl group. Among the chromophores of this type, examples that may be mentioned include chromophores derived from dyes chosen from acridone, benzoquinone, anthraquinone, naphthoquinone, benzanthrone, anthranthrone, pyranthrone, pyrazolanthrone, pyrimidinoanthrone, flavanthrone, indanthrone, flavone, (iso)violanthrone, isoindolinone, benzimidazolone, isoquinolinone, anthrapyridone, pyrazoloquinazolone, perinone, quinacridone, quinophthalone, indigoid, thioindigo, naphthalimide, anthrapyrimidine, diketopyrrolopyrrole and coumarin dyes.

Examples of colored polymers that may be mentioned include:

| | |
|---|---|
| Poly S-119 (orange) | 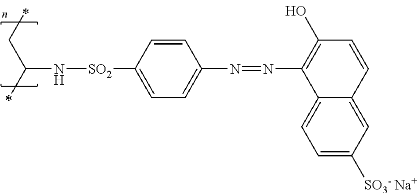 |
| Poly R-478 (violet) | 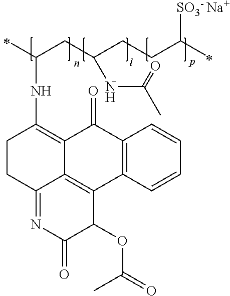 |
| PAZO (yellow) | 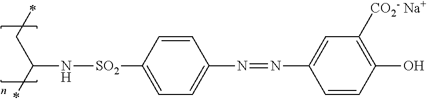 |
| Poly Black-863 (black) | 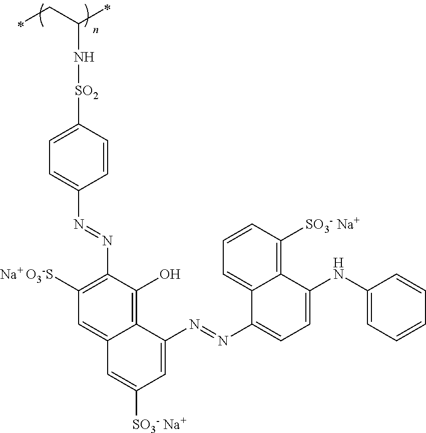 |
| Poly Y-606 (yellow) | 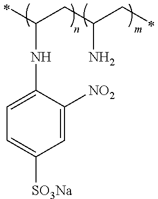 |
| Poly Y-636 (yellow) | 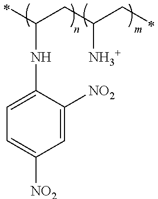 |
| Poly R-480 (red) | 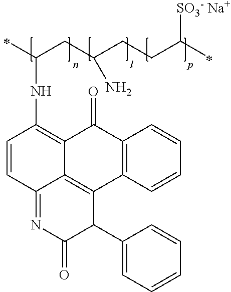 |
| Poly T-128 (yellow) | 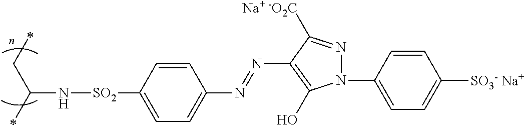 |
| Poly[methyl methacrylate- co- (fluorescein O-methacylate)] (fluorescent yellow) | 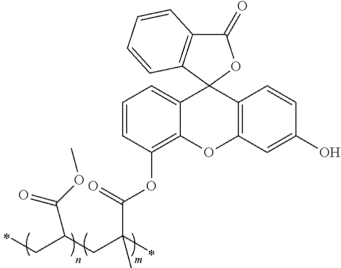 |
| Poly (Disperse Red 13 methacrylate) (red) | 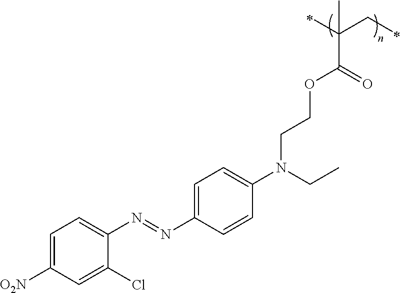 |

Examples of anionic chromophores that may be mentioned include chromophores substituted with at least one sulfonate, carboxylate, phosphate, phosphonate or sulfate group. Examples that may be mentioned include radicals derived from acidic nitro direct dyes, acidic azo dyes, acidic azine dyes, acidic triarylmethane dyes, acidic indoamine dyes and non-quinone acidic natural dyes.

Anionic colored polyelectrolytes that may be mentioned include those described in U.S. Pat. No. 4,911,731, U.S. Pat. No. 6,306,182, EP 852 943, EP 747 036, U.S. Pat. No. 4,381,260, U.S. Pat. No. 4,314,808, U.S. Pat. No. 4,144,252 and U.S. Pat. No. 4,051,138.

By way of example, the anionic colored polymer corresponds to the formula:

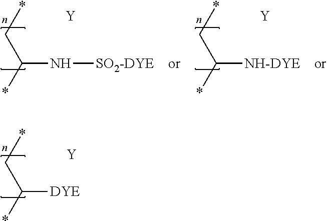

in which DYE is an anionic chromophoric radical and Y is the counterion.

The amount of colored polyelectrolytes in the composition applied to the fibers is generally between 0.01% and 20% and preferably between 0.1% and 5%. According to one particular embodiment, the composition is an aqueous composition.

For the purposes of the present invention, the term “oil” means a substance that is liquid at room temperature, for example 25° C., and at atmospheric pressure, and that is water-insoluble.

For the purposes of the present invention, the term “water-insoluble” refers to a substance that has a solubility in pure water of less than 1% at 25° C. and at atmospheric pressure.

The oils used in the present invention have a dynamic viscosity at 25° C. of less than 1 Pa·s (1000 cps) and preferably between 10⁻³ and 0.1 Pa·s (1 and 100 cps). The dynamic viscosity is measured at 25° C. with a shear rate of 100 s⁻¹, for example using the machine referenced RM 180 Rheomat from the company Mettler.

The oils that may be used in the present invention are preferably chosen from plant oils, mineral oils, synthetic oils, liquid fatty acid esters and liquid fatty acids.

Among the plant oils that may be used in the present invention, mention may be made especially of sweet almond oil, avocado oil, castor oil, olive oil, jojoba oil, sunflower oil, wheatgerm oil, sesame seed oil, groundnut oil, grapeseed oil, soybean oil, rapeseed oil, safflower oil, coconut oil, corn oil, hazelnut oil, shea butter, palm oil, apricot kernel oil and beauty-leaf oil.

Examples of mineral oils are liquid paraffin and liquid petroleum jelly.

The synthetic oils may be chosen especially from polydecenes, squalane, poly(α-olefins), for instance isododecane or isohexadecane, transesterified plant oils and fluoro oils.

It is also possible to use fatty acid esters, for instance the compounds of formula RaCOORb in which Ra represents a higher fatty acid residue containing from 6 to 29 carbon atoms and Rb represents a hydrocarbon-based chain containing from 3 to 30 carbon atoms, such as purcellin oil (stearyl octanoate), isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or 2-octyldodecyl lactate.

Liquid fatty acids, for instance oleic acid, may also be used.

The oils that are particularly preferred are liquid fatty acids, liquid fatty esters and plant oils.

The oil(s) as defined above is (are) preferably present in an amount of between 0.01% and 95% by weight and even more preferentially in an amount of between 1% and 60% by weight relative to the weight of the composition.

The alkylpolyglucoside surfactants present in the composition are surfactants that are well known in the prior art. These surfactants may be more particularly represented by the following general formula:

R₁O—(R₂O)ₜ(G)ᵥ

in which R₁ represents a linear or branched alkyl and/or alkenyl radical containing from 8 to 24 carbon atoms approximately, an alkylphenyl radical in which the linear or branched alkyl radical contains from 8 to 24 carbon atoms, R₂ represents an alkylene radical containing from 2 to 4 carbon atoms approximately, G represents a sugar unit containing from 5 to 6 carbon atoms, t denotes a value ranging from 0 to 10 and preferably from 0 to 4, preferably 0 to 4, and v denotes a value ranging from 1 to 15.

According to one particular embodiment, the alkylpolyglucoside surfactants are compounds of formula (II) in which R₁ more particularly denotes a linear or branched saturated or unsaturated alkyl radical containing from 8 to 18 carbon atoms, t denotes a value ranging from 0 to 3 and even more particularly equal to 0, and G may denote glucose, fructose or galactose, preferably glucose. The degree of polymerization, i.e. the value of v in formula (II), may range from 1 to 15 and preferably from 1 to 4. The mean degree of polymerization is more particularly between 1 and 2.

The glucoside bonds between the sugar units are of 1-6 or 1-4 type, preferably 1-4 type.

Compounds of formula (II) are especially represented by the products sold by the company Cognis under the names Plantaren® (600 CS/U, 1200 and 2000) or Plantacare® (818, 1200 and 2000). It is also possible to use the products sold by the company SEPPIC under the names Triton CG110 (or Oramix CG 10) and Triton CG312 (or Oramix® NS10), the products sold by the company BASF under the name Lutensol GD 70 or those sold by the company Chem Y under the name AG10 LK.

Mention may also be made of the C₈/C₁₆ alkyl 1,4-polyglucoside as a 53% aqueous solution sold by Cognis under the reference Plantacare® 818 UP.

The amount of alkylpolyglucoside surfactants present in the composition is generally between 0.6% and 30% and preferentially between 0.5% and 10% relative to the total weight of the composition.

For the purposes of the present patent application, the term “fixing polymer” means a polymer that allows the shaping or retention of the shape of keratin fibers such as the hair.

The term “non-associative polymer” means a polymer not containing any hydrophobic chains, especially carbon-based chains containing more than 10 carbon atoms.

Any cationic, amphoteric or nonionic fixing polymer or mixture thereof used in the art may be used in the compositions according to the present patent application. Anionic fixing polymers are anionic polymers containing a carboxylic function.

The fixing polymers are soluble in the cosmetically acceptable medium.

The anionic fixing polymers have a number-average molecular mass of approximately between 500 and 5 000 000.

The carboxylic groups are provided by unsaturated monocarboxylic or dicarboxylic acid monomers such as those corresponding to the formula:

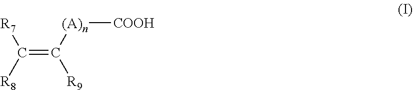

in which n is an integer from 0 to 10, A₁ denotes a methylene group, optionally connected to the carbon atom of the unsaturated group, or to the neighboring methylene group when n is greater than 1, via a heteroatom such as oxygen or sulfur, R₇ denotes a hydrogen atom or a phenyl or benzyl group, R₈ denotes a hydrogen atom or a lower alkyl or carboxyl group, R₉ denotes a hydrogen atom, a lower alkyl group or a —CH₂—COOH, phenyl or benzyl group.

In the abovementioned formula, a lower alkyl group preferably denotes a group having 1 to 4 carbon atoms and in particular methyl and ethyl groups.

The anionic fixing polymers containing carboxylic groups that are preferred according to the invention are:
A) acrylic or methacrylic acid copolymers, or salts thereof and in particular the products sold under the names Ultrahold® by the company BASF, the copolymers of acrylic acid and of acrylamide sold in the form of their sodium salt under the names Reten 421, 423 or 425 by the company Hercules, the sodium salts of polyhydroxycarboxylic acids;
B) copolymers of acrylic or methacrylic acids with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent 1 222 944 and German patent application 2 330 956, the copolymers of this type comprising an optionally N-alkylated and/or N-hydroxyalkylated acrylamide unit in their chain as described in particular in Luxembourg patent applications 75370 and 75371 or sold under the name Quadramer by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name Acrylidone® LM and methacrylic acid/ethyl acrylate/tert-butyl acrylate terpolymers such as the product sold under the name Luvimer® 100 P by the company BASF.
Mention may also be made of methacrylic acid/acrylic acid/ethyl acrylate/methyl methacrylate copolymers as an aqueous dispersion, sold under the name Amerhold® DR 25 by the company Amerchol;
C) crotonic acid copolymers such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those containing at least 5 carbon atoms, it being possible for these polymers optionally to be grafted or crosslinked, or alternatively another vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products falling into this class are the resins 28-29-30, 26-13-14 and 28-13-10 sold by the company National Starch;
D) copolymers of C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described in particular in U.S. Pat. No. 2,047,398, U.S. Pat. No. 2,723,248 and U.S. Pat. No. 2,102,113 and GB patent GB 839 805. Commercial products are in particular those sold under the names Gantrez® AN or ES by the company ISP;
copolymers comprising (i) one or more maleic, citraconic or itaconic anhydride units and (ii) one or more monomers chosen from allylic or methallylic esters optionally comprising one or more acrylamide, methacrylamide, α-olefin, acrylic or methacrylic ester, acrylic or methacrylic acid or vinylpyrrolidone groups in their chain,
   the anhydride functions of these copolymers optionally being monoesterified or monoamidated.

These polymers are described, for example, in French patents FR 2 350 384 and FR 2 357 241 by the Applicant;
E) polyacrylamides comprising carboxylate groups.
The homopolymers and copolymers comprising sulfonic groups are polymers comprising vinylsulfonic, styrenesulfonic, naphthalenesulfonic or acrylamido-alkylsulfonic units.

These polymers can be chosen especially from:
polyvinylsulfonic acid salts having a molecular mass of approximately between 1000 and 100 000, and also the copolymers with an unsaturated comonomer such as acrylic or methacrylic acids and their esters, and also acrylamide or its derivatives, vinyl ethers and vinylpyrrolidone;
polystyrenesulfonic acid salts, such as the sodium salts sold for example under the names Flexan® 500 and Flexan® 130 by National Starch. These compounds are described in patent FR 2 198 719;
polyacrylamidesulfonic acid salts, such as those mentioned in U.S. Pat. No. 4,128,631 and more particularly polyacrylamidoethylpropanesulfonic acid sold under the name Cosmedia Polymer HSP 1180 by the company Henkel.

As another anionic fixing polymer that can be used according to the invention, mention may be made of the branched block anionic polymer of the (meth)acrylic acid, (meth)acrylic acid alkyl ester and allyl methacrylate anionic copolymer type known as MAP acrylate/allyl methacrylate copolymer, sold under the name Fixate G-100 by the company Noveon.

According to the invention, the anionic fixing polymers are preferably chosen from acrylic acid copolymers, such as the acrylic acid/ethyl acrylate/N-tert-butylacrylamide terpolymers sold especially under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold especially under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez® by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF, the vinyl acetate/crotonic acid copolymers sold under the name Luviset CA 66 by the company BASF, the vinyl acetate/crotonic acid copolymers grafted with polyethylene glycol sold under the name Aristoflex® A by the company BASF, and the polymer sold under the name Fixate G-100 by the company Noveon.

Among the anionic fixing polymers mentioned above, it is more particularly preferred in the context of the present invention to use the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer®MAEX or MAE by the company BASF, the vinylpyrrolidone/acrylic acid/lauryl methacrylate terpolymers sold under the name Acrylidone® LM by the company ISP, and the polymer sold under the name Fixate G-100 by the company Noveon.

The cationic fixing film-forming polymers that may be used according to the present invention are preferably chosen from polymers comprising primary, secondary, tertiary and/or quaternary amine groups forming part of the polymer chain or directly attached thereto, and having a molecular weight of between 500 and about 5 000 000 and preferably between 1000 and 3 000 000.

Among these polymers, mention may be made more particularly of the following cationic polymers:
(1) homopolymers or copolymers derived from acrylic or methacrylic esters or amides, and comprising at least one of the units of the following formulae:

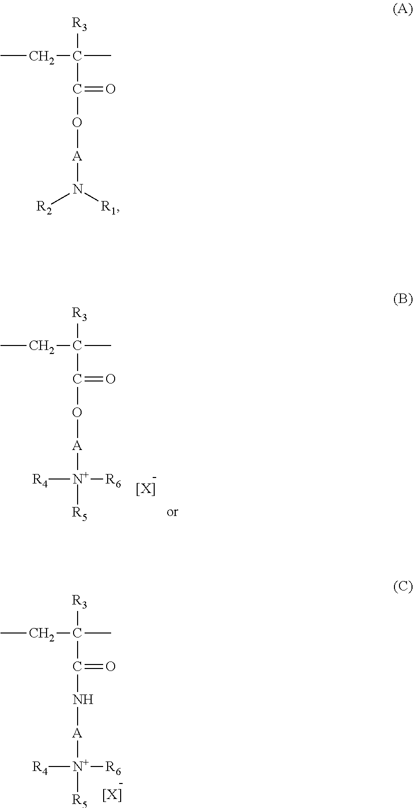

in which:
R₃ denotes a hydrogen atom or a CH₃ radical;
A is a linear or branched alkyl group comprising 1 to 6 carbon atoms or a hydroxyalkyl group comprising 1 to 4 carbon atoms;
R₄, R₅ and R₆, which may be identical or different, represent an alkyl group having from 1 to 8 carbon atoms or a benzyl group;
R₁ and R₂, which may be identical or different, each represent a hydrogen atom or an alkyl group having from 1 to 6 carbon atoms;
X denotes a methosulfate anion or a halide such as chloride or bromide.

The copolymers of the family (1) also contain one or more units derived from comonomers that may be chosen from the family of acrylamides, methacrylamides, diacetone acrylamides, acrylamides and methacrylamides substituted on the nitrogen with lower (C₁₋₄)alkyl groups, groups derived from acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the one sold under the name Hercofloc® by the company Hercules,
copolymers of acrylamide and of methacryloyloxyethyl-trimethylammonium chloride, described, for example, in patent application EP-A-080 976 and sold under the name Bina Quat P 100 by the company Ciba Geigy,
copolymers of acrylamide and of methacryloyloxyethyl-trimethylammonium methosulfate, such as the product sold under the name Reten by the company Hercules,
quaternized or non-quaternized vinylpyrroli-done/dialkylaminoalkyl acrylate or methacrylate copolymers, such as the products sold under the name Gafquat® by the company ISP, such as, for example, Gafquat® 734 or Gafquat® 755, or alternatively the products known as Copolymer® 845, 958 and 937. These polymers are described in detail in French patents 2 077 143 and 2 393 573,
dimethylaminoethyl methacrylate/vinylcaprolactam/-vinylpyrrolidone terpolymers, such as the product sold under the name Gaffix VC 713 by the company ISP, and
quaternized vinylpyrrolidone/dimethylaminopropyl-methacrylamide copolymers, such as the product sold under the name Gafquat® HS 100 by the company ISP;
(2) cationic polysaccharides, preferably containing quaternary ammonium, such as those described in U.S. Pat. Nos. 3,589,578 and 4,031,307, such as guar gums containing trialkylammonium cationic groups. Such products are sold in particular under the trade names Jaguar C13S, Jaguar C15 and Jaguar C17 by the company Meyhall;
(3) quaternary copolymers of vinylpyrrolidone and of vinylimidazole;
(4) cationic cellulose derivatives such as copolymers of cellulose or of cellulose derivatives grafted with a water-soluble monomer comprising a quaternary ammonium, and disclosed in particular in U.S. Pat. No. 4,131,576, such as hydroxyalkylcelluloses, for instance hydroxymethyl-, hydroxyethyl- or hydroxy-propylcelluloses grafted in particular with a methacryloyloxyethyltrimethylammonium, methacrylamido-propyltrimethylammonium or dimethyldiallylammonium salt.

The commercial products corresponding to this definition are, more particularly, the products sold under the name Celquat L 200 and Celquat H 100 by the company National Starch.

The amphoteric fixing polymers that can be used in accordance with the invention can be chosen from polymers comprising units B and C distributed randomly in the polymer chain, in which B denotes a unit derived from a monomer comprising at least one basic nitrogen atom and C denotes a unit derived from an acid monomer comprising one or more carboxylic or sulfonic groups, or alternatively B and C can denote groups derived from carboxybetaine or sulfobetaine zwitterionic monomers;

B and C can also denote a cationic polymer chain comprising primary, secondary, tertiary or quaternary amine groups, in which at least one of the amine groups bears a carboxylic or sulfonic group connected via a hydrocarbon group or alternatively B and C form part of a chain of a polymer containing an α,β-dicarboxylic ethylene unit in which one of the carboxylic groups has been made to react with a polyamine comprising one or more primary or secondary amine groups.

The amphoteric fixing polymers corresponding to the definition given above that are more particularly preferred are chosen from the following polymers:
(1) copolymers containing acidic vinyl and basic vinyl units, such as those resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamides and -acrylamides. Such compounds are described in U.S. Pat. No. 3,836,537;
(2) polymers comprising units derived from:
   a) at least one monomer chosen from acrylamides and methacrylamides substituted on the nitrogen atom with an alkyl group,
   b) at least one acidic comonomer containing one or more reactive carboxylic groups, and
   c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacryl-amides.

The acidic comonomers are chosen more particularly from acrylic acid, methacrylic acid, crotonic acid, itaconic acid, maleic acid and fumaric acid and alkyl monoesters, having 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer® or Lovocryl® 47 by the company National Starch, are particularly used;
(3) crosslinked and acylated polyamino amides partially or totally derived from polyamino amides of general formula:

CO—R₁₀—CO—Z (II)

in which R₁₀ represents a divalent group derived from a saturated dicarboxylic acid, a mono- or dicarboxylic aliphatic acid containing an ethylenic double bond, an ester of a lower alkanol, having 1 to 6 carbon atoms, of these acids, or a group derived from the addition of any one of said acids to a bis(primary) or bis(secondary)amine, and Z denotes a group derived from a bis(primary), mono- or bis(secondary) polyalkylene-polyamine and preferably represents:
a) in proportions of from 60 to 100 mol %, the group:

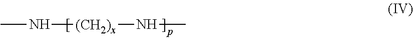

where x=2 and p=2 or 3, or alternatively x=3 and p=2
this group being derived from diethylenetriamine, from triethylenetetraamine or from dipropylenetriamine;
b) in proportions of from 0 to 40 mol %, the group (IV) above in which x=2 and p=1 and which is derived from ethylenediamine, or the group derived from piperazine:

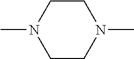

c) in proportions of from 0 to 20 mol %, the —NH(CH₂)₆—NH— group derived from hexamethylenediamine, these polyamino amides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation are preferably propane sultone or butane sultone, the salts of the acylating agents are preferably the sodium or potassium salts;
(4) polymers comprising zwitterionic units of formula:

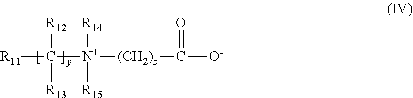

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, a methyl, ethyl or propyl group, R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.

By way of example, mention may be made of the copolymers of methyl methacrylate/methyl dimethylcarboxymethylammonioethyl methacrylate such as the product sold under the name Diaformer 2301 by the company Sandoz;
(5) polymers corresponding to the general formula (V) are described, for example, in French patent 1 400 366 and are constituted of units:

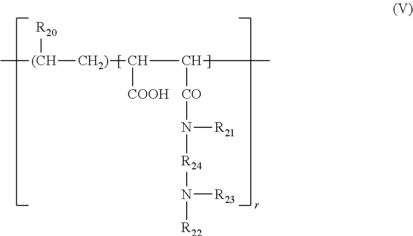

in which R₂₀ represents a hydrogen atom, a CH₃O, CH₃CH₂O or phenyl group, R₂₁ denotes a hydrogen atom or a lower alkyl group such as methyl or ethyl, R₂₂ denotes a hydrogen atom or a C₁₋₆ lower alkyl group such as methyl or ethyl, R₂₃ denotes a C₁₋₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above;
(6) polymers derived from the N-carboxyalkylation of chitosan, such as N-carboxymethylchitosan or N-carboxybutylchitosan sold under the name Evalsan by the company Jan Dekker;
(7) amphoteric polymers of the type -D-X-D-X-chosen from:
   a) polymers obtained by the action of chloroacetic acid or sodium chloroacetate on compounds comprising at least one unit of formula:

-D-X-D-X-D- (VI)

where D denotes a group

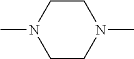

and X denotes the symbol E or E′, E or E′, which may be identical or different, denote a divalent group that is an alkylene group with a straight or branched chain containing up to 7 carbon atoms in the main chain, which is unsubstituted or substituted with hydroxyl groups and which can comprise, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;
b) polymers of formula:

-D-X-D-X— (VI′)

where D denotes a group

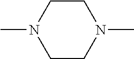

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate;
(8) (C₁-C₅)alkyl vinyl ether/maleic anhydride copolymers partially modified by semiamidation with an N,N-dialkylaminoalkylamine such as N,N-dimethylamino-propylamine or by semiesterification with an N,N-dialkylaminoalkanol. These copolymers can also comprise other vinyl comonomers such as vinylcapro-lactam.

Among the amphoteric fixing polymers described above, the ones that are most particularly preferred according to the invention are those of family (3), such as the copolymers whose CTFA name is octylacryl-amide/acrylates/butylaminoethyl methacrylate copolymer, such as the products sold under the names Amphomer®, Amphomer® LV 71 or Lovocryl® 47 by the company National Starch and those of family (4) such as the copolymers of methyl methacrylate/methyl dimethylcarboxymethyl-ammonioethyl methacrylate, sold, for example, under the name Diaformer 2301 by the company Sandoz.

The nonionic fixing polymers that may be used according to the present invention are chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene; copolymers of vinyl acetate and of maleic ester, for example of dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl(meth)acrylates; mention may be made of the products sold under the name CJ 0601 B by the company Rohm & Haas;
styrene homopolymers;
styrene copolymers, for instance copolymers of styrene and of an alkyl(meth)acrylate, such as the products Mowilith® LDM 6911, Mowilith® DM 611 and Mowilith® LDM 6070 sold by the company Hoechst, and the products Rhodopas® SD 215 and Rhodopas® DS 910 sold by the company Rhone-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers such as vinylpyr-rolidone homopolymers, the polyvinylcaprolactam sold under the name Luviskol® Plus by the company BASF; and
vinyllactam copolymers such as a polyvinyl-pyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyr-rolidone/vinyl acetate/vinyl propionate) terpolymers, for instance the product sold under the name Luviskol® VAP 343 by the company BASF.

The alkyl groups of the nonionic polymers mentioned above preferably contain from 1 to 6 carbon atoms.

Functionalized or non-functionalized, silicone or non-silicone, cationic, nonionic, anionic or amphoteric polyurethanes or mixtures thereof may also be used as fixing polymers.

As polyurethanes that are particularly suitable for the present invention, mention may be made of polyurethane/polysiloxane block anionic polymers such as the products sold under the names Luviset Pur® and Luviset® Si-Pur by the company BASF.

In a particularly advantageous manner, the compositions according to the invention comprise, as nonionic fixing polymer, a vinyllactam copolymer, such as polyvinylpyrrolidones, a poly(vinylpyrrolidone/vinyl-lactam) copolymer, a poly(vinylpyrrolidone/vinyl acetate) copolymer or a poly(vinylpyrrolidone/vinyl acetate/vinyl propionate) terpolymer.

According to one particular embodiment, the cosmetic composition according to the invention comprises, as fixing polymer, an anionic or nonionic polymer, preferably a nonionic polymer.

The concentration of fixing polymer(s) used in the compositions according to the present invention is between 0.1% and 20% and preferably between 0.5% and 10% by weight relative to the total weight of the composition.

### Organosilicon Compounds

Organosilicon compounds that may especially be mentioned include silicones and silanes.

The silicones that may be used in the composition according to the invention may be soluble or insoluble in said composition.

The insoluble silicones are especially dispersed in the composition in the form of particles generally having a number-average size of between 2 nanometers and 100 micrometers and preferably between 20 nanometers and 20 micrometers (measured with a granulometer).

The polyorganosiloxanes are defined in greater detail in Walter Noll's “Chemistry and Technology of Silicones” (1968) Academic Press. They can be volatile or nonvolatile.

Thus, the composition according to the invention may comprise at least one volatile polyorganosiloxane, chosen from those having a boiling point of between 60° C. and 260° C., and even more particularly from:
(i) cyclic silicones containing from 4 to 5 silicon atoms. These are, for example, octamethylcyclotetrasiloxane sold in particular under the name Volatile Silicone 7207 by Union Carbide or Silbione 70045 V 2 by Rhodia, decamethylcyclo-pentasiloxane sold under the name Volatile Silicone 7158 by Union Carbide, and Silbione 70045 V 5 by Rhodia, and mixtures thereof.

Mention may also be made of cyclocopolymers of the dimethylsiloxane/methylalkylsiloxane type, such as Volatile Silicone FZ 3109 sold by the company Union Carbide, having the chemical structure:

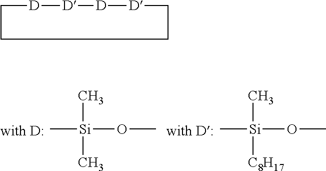

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile silicones containing 2 to 5 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

The siloxanes that are useful may also be chosen from the organosiloxanes having the formula:

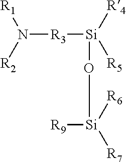

in which:
R₁, R₂, R₃, R₅ and R₆ are defined as above;
R′₄ represents a halogen or a group OR₁₁;
R₇ represents a halogen or a group OR₁₀ or R″₁;
R₉ represents a halogen or a group OR_{B}, R″₂ or R₃NR₁R₂;
R″₁, R″₂, R₈, R₁₀ and R₁₁ represent a saturated or unsaturated, linear or branched hydrocarbon-based group optionally bearing additional chemical groups such as basic solubilizing groups;
R₁₁, R₁₀ and R₈ also possibly denoting hydrogen.

Preferably, R″₁, R″₂, R₈ or R₁₀ and R₁₁ represent a C₁-C₁₂ alkyl group, a C₅-C₁₄ aryl group, a (C₁-C₈)alkyl(C₅-C₁₄)aryl group and a (C₅-C₁₄)aryl(C₁-C₈)alkyl group.

At least one of the groups R₆, R₇ and R₉ denotes a halogen or a group OR′″, OR₁₀ or OR₈.

The halogen is preferably chlorine.

The silicones that are particularly preferred in the invention are polydimethylsiloxanes such as polydimethylsiloxanes containing trimethylsilyl end groups.

Organosilicon compounds that may especially be mentioned are silanes.

For example, the silanes of formula (1)

(R₁₀)ₓ(R₂)_{y}Si (A)ₚ-[NR₃-(A′)_{p′}]_{q}-[NR′₃-(A″)_{p″}]_{q′}-Si—(R′₂)_{y}(OR′₁)_{x′} (1)

in which
R₁, R₂, R′₁, and R′₂ each independently represent a saturated or unsaturated, linear or branched hydrocarbon-based chain, optionally containing one or more heteroatoms, optionally interrupted or substituted with one or more groups chosen from ether, ester, amine, amide, carboxyl, hydroxyl and carbonyl groups,
x is an integer ranging from 1 to 3,
y=3−x,
x′ is an integer ranging from 1 to 3,
y′=3−x′,
p=0 or 1,
p′=0 or 1,
p″=0 or 1,
q=0 or 1,
q′=0 or 1,
it being understood that at least q or q′ is other than zero,
A, A′ and A″ each independently represent a linear or branched C₁-C₂₀ divalent alkylene radical,
R₃ and R′₃ each independently represent a hydrogen atom or a saturated or unsaturated, linear or branched hydrocarbon-based chain optionally containing one or more heteroatoms, optionally interrupted or substituted with one or more ether or ester of a C₁-C₂₀ alcohol, amine, carboxyl, alkoxysilane, C₆-C₃₀ aryl, hydroxyl or carbonyl group, or a heterocyclic or non-heterocyclic aromatic ring, optionally substituted with one or more ester of a C₃-C₂₀ alcohol, amine, amide, carboxyl, alkoxysilane, hydroxyl, carbonyl or acyl group,
said cosmetic composition also comprising at least one cosmetic agent chosen from polymers with a molecular weight at least equal to 500, surfactants and non-silicone fatty substances.

According to one particular embodiment, the silanes of formula (1) are such that R₁═R′₁, R₂═R′₂, x=x′, y=y′, p=p′, A=A′, q=1 and q′=0.

In particular, mention may be made of the compounds of formula (1) in which R₁, R₂, R′₁ and R′₂, which may be identical or different, represent a C₁-C₄ alkyl. According to another embodiment, p=p′=1 and A and A′, which may be identical or different, represent a linear C₁-C₄ alkylene.

Mention may also be made especially of the compounds with R₃ representing hydrogen.

Examples that may be mentioned include:
bis[3-(triethoxysilyl)propyl]amine, of formula:

(CH₃CH₂O)₃—Si—(CH₂)₃—NH—(CH₂)₃—Si—(OCH₂CH₃)₃,
bis[trimethoxysilylpropyl]amine, of formula

(CH₃O)₃—Si—(CH₂)₃—NH—(CH₂)₃—Si—(OCH₃)₃,
bis[methyldiethoxysilylpropyl]amine, of formula:

(CH₃CH₂O)₂CH₃—Si—(CH₂)₃—NH—(CH₂)₃—Si—CH₃(OCH₂CH₃)₂,
bis[(3-trimethoxysilyl)propyl]ethylene diamine, of formula:

(CH₃O)₃—Si—(CH₂)₃—NH—(CH₂)₂—NH—(CH₂)₃—Si—(OCH₃)₃.

Among these compounds, bis[3-(triethoxysilyl)propyl]-amine is preferred.

The silanes may also be represented by formula (2) below:

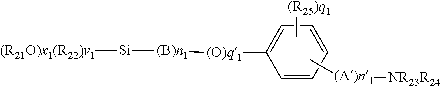

in which
R₂₁ and R₂₂ each independently represent a saturated or unsaturated, linear or branched hydrocarbon-based chain, optionally containing one or more heteroatoms, optionally interrupted or substituted with one or more groups chosen from ether, ester, amine, amide, carboxyl, hydroxyl and carbonyl groups,
x₁=2 or 3,
y₁=3−x,
n₁=0 or 1,
n′₁═0 or 1,
A and A′ each independently represent a linear or branched divalent C₁-C₂₀ alkylene radical,
R₂₃ and R₂₄ each independently represent hydrogen or a saturated or unsaturated, linear or branched hydrocarbon-based chain optionally containing one or more heteroatoms, optionally interrupted or substituted with one or more ether, ester of a C₁-C₂₀ alcohol, amine, carboxyl, alkoxysilane, C₆-C₃₀ aryl, hydroxyl or carbonyl group, or a heterocyclic or non-heterocyclic aromatic ring, optionally substituted with one or more ester of a C₁-C₂₀ alcohol, amine, amide, carboxyl, alkoxysilane, hydroxyl, carbonyl or acyl group,
q₁ is an integer ranging from 0 to 4,
q′₁=0 or 1,
the group(s) R₂₅ each independently represent hydrogen or a saturated or unsaturated, linear or branched hydrocarbon-based chain optionally containing one or more heteroatoms, optionally interrupted or substituted with one or more ether, ester of a C₁-C₂₀ alcohol, amine, carboxyl, alkoxysilane, C₆-C₃₀ aryl, hydroxyl or carbonyl group, or a heterocyclic or non-heterocyclic aromatic ring, optionally substituted with one or more ester of a C₁-C₂₀ alcohol, amine, amide, carboxyl, alkoxysilane, hydroxyl, carbonyl or acyl group,
said composition also comprising at least one compound chosen from hydroxylated solvents, polymers, surfactants and fatty substances.

According to one embodiment, R₂₁ is a C₁-C₄ alkyl.

According to another embodiment, x₁=3. According to a different embodiment, n₁=n′₁=1. q₁ and q′₁ may be equal to 0.

R₂₃ and R₂₄ may independently represent hydrogen or a group chosen from C₁-C₄ alkyl, C₁-C₄ hydroxyalkyl and C₁-C₄ aminoalkyl groups.

Examples of compounds of formula (2) that may be mentioned include:
3-(m-aminophenoxy)propyltrimethoxysilane, of formula:

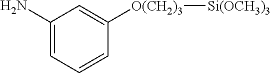

p-aminophenyltrimethoxysilane, of formula:

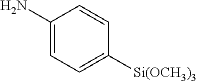

N-(2-aminoethylaminomethyl)phenethyltrimethoxysilane, of formula:

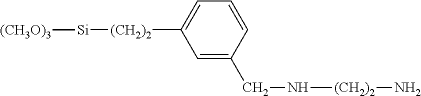

Silanes compounds that may also be mentioned include the organosilicon compounds of formula:

(R″₁O)_{x″}(R″₂)_{y″}Si-(A)_{p″}CH═O (3)

or of formula

(R″₁O)_{x″}(R″₂)_{y″}—Si-(A)_{p″}—CH(OR″₃)(OR″₄) (4)

in which
R″₁, which may be identical or different, and R″₂ each independently represent a saturated or unsaturated, linear or branched hydrocarbon-based chain, optionally containing one or more heteroatoms, optionally interrupted or substituted with one or more groups chosen from ether, ester, amine, amide, carboxyl, hydroxyl and carbonyl groups,
x″=2 or 3,
y″=3−x′,
A represents a linear or branched divalent C₁-C₂₀ alkylene radical, optionally interrupted or substituted with one or more ester of a C₁-C₃₀ alcohol, amine, carboxyl, alkoxy silane, C₆-C₃₀ aryl, hydroxyl or carbonyl group,
p″═0 or 1,
R″₃ and R″₄ each independently represent a saturated or unsaturated, linear or branched hydrocarbon-based chain optionally containing one or more heteroatoms, optionally interrupted or substituted with one or more ether, ester of a C₁-C₂₀ alcohol, amine, carboxyl, alkoxysilane, C₆-C₃₀ aryl, hydroxyl or carbonyl group, or a heterocyclic or non-heterocyclic aromatic ring, optionally substituted with one or more ester of a C₁-C₂₀ alcohol, amine, amide, carboxyl, alkoxysilane, hydroxyl, carbonyl or acyl group.

According to a first embodiment, R″₁ and R″₂ are chosen from C₁-C₄ alkyls. According to a second embodiment, p″=1.

According to another embodiment, A is a linear C₁-C₄ alkylene.

Examples of silane compounds of formula (3) or (4) that may be mentioned include:
triethoxysilylbutyraldehyde, of formula:

(CH₃CH₂O)₃—Si—(CH₂)₃—CH═O
triethoxysilylundecanal, of formula:

(CH₃CH₂O)₃—Si—(CH₂)₁₀—CH═O
triethoxysilylundecanal ethylene glycol acetal, of formula:

(CH₃CH₂O)₃—Si—(CH₂)₁₀—CH(OCH₂)₂

According to one particularly preferred embodiment, the silane compound is chosen from triethoxysilylbutyr-aldehyde.

Other useful organosilanes correspond to the formula:

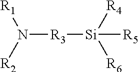

in which:
R₄ represents a halogen or a group OR′ or R′₁;
R₅ represents a halogen or a group OR″ or R′₂;
R₆ represents a halogen or a group OR′″ or R′₃;
and R₁, R₂, R₃, R′, R″, R′″, R′₁, R′₂ and R′₃ represent, independently of each other, a saturated or unsaturated, linear or branched hydrocarbon-based group optionally bearing additional chemical groups such as acid or amine groups, R₁, R₂, R′, R″ and R′″ also possibly denoting hydrogen, and
at least two of the groups R₄, R₅ and R₆ being other than groups R′₁, R′₂ and R′₃.

Preferably, R₁, R₂, R′, R″ and R′″, R′₁, R′₂ and R′₃ represent a C₁-C₁₂ alkyl group, a C₅-C₁₄ aryl group, a (C₁-C₈)alkyl(C₅-C₁₄)aryl group and a (C₅-C₁₄)aryl(C₁-C₈)alkyl group; and R₃ is preferably a C₁-C₁₂ alkylene group, a C₅-C₁₄ arylene group, a (C₁-C₈)alkyl(C₅-C₁₄)arylene group and a (C₅-C₁₄)aryl(C₁-C₈)alkylene group.

The preferred organosilicon compounds are 3-amino-propyltriethoxysilane, 3-aminopropylmethyldiethoxy-silane and 3-[bis(hydroxyethyl)amino]propyltriethoxy-silane.

The compound that is particularly preferred is 3 aminopropyltriethoxysilane.

The organosilicon compounds presented above is (are) preferably present in an amount of between 0.01% and 95% by weight and even more preferentially in an amount of between 1% and 60% by weight relative to the weight of the composition.

Optionally, the composition may contain a silicone oil. Examples that may especially be mentioned include polydimethylsiloxanes (PDMS), phenyl polyorgano-siloxanes such as phenyl trimethicones, phenyltri-methylsiloxydiphenylsiloxanes, diphenylmethyldimethyl-trisiloxanes, diphenyl dimethicones, phenyl dimethicones and polymethylphenylsiloxanes, which are optionally fluorinated; polysiloxanes modified with fatty acids, fatty alcohols or polyoxyalkylenes, fluoro silicones and perfluoro silicone oils.

Mention may be made, for example, of polydimethyl-siloxanes, polymethylphenylsiloxanes, silicones bearing polyoxyalkylene, in particular polyoxyethylene or copoly(oxyethylene/oxypropylene), blocks or grafts, such as dimethicone copolyols, silicones bearing both hydrocarbon-based hydrophobic groups (for example C₂-C₃₀ alkyl groups) and polyoxyethylene or copoly(oxyethylene/oxypropylene) blocks or grafts, such as alkyldimethicone copolyols, silicones bearing fluoro or perfluoro groups, such as perfluoroalkyl polydimethylsiloxanes and perfluoroalkyl polymethyl-phenylsiloxanes, and mixtures thereof.

These silicone oils may optionally comprise alkyl or alkoxy groups at the end of a silicone chain or pendent thereon.

According to one variant, the composition containing the colored polymer contains an organic and/or mineral salt. An organic salt that may be mentioned is sodium citrate. Mineral salts that may be mentioned include sodium chloride, ammonium sulfate, magnesium chloride and calcium chloride.

The amount of organic or mineral salts is generally between 10⁻⁴ and 2 mol/l and preferably between 10⁻³ and 1 mol/l. According to one particularly preferred embodiment, the amount of salts is between 10⁻² and 1 mol/l.

The composition that is useful in the process of the present invention may comprise water or a mixture of water and of at least one non-oily organic solvent to dissolve the compounds that are not sufficiently water-soluble. Examples of organic solvents that may be mentioned include C₁-C₄ lower alkanols, such as ethanol and isopropanol; polyols and polyol ethers, for instance 2-butoxyethanol, propylene glycol, propylene glycol monomethyl ether, diethylene glycol monomethyl ether and monoethyl ether, and also aromatic alcohols, for instance benzyl alcohol or phenoxyethanol, and mixtures thereof.

The non-oily solvents are preferably present in proportions preferably between 0.5% and 80% by weight approximately, and even more preferentially between 5% and 60% by weight approximately, relative to the total weight of the composition.

The colored polymers are preferably chosen from colored polymers that are soluble or partially soluble in water at room temperature.

The compositions that are useful in the process of the invention may also contain various adjuvants conventionally used in hair dye compositions, such as mineral or organic thickeners, and in particular anionic, cationic, nonionic and amphoteric polymeric associative thickeners, antioxidants, penetrants, sequestrants, fragrances, buffers, dispersants, conditioning agents, ceramides, preserving agents and opacifiers.

The compositions that are useful in the process of the invention may also contain at least one thickening polymer, also known as “rheology modifiers”.

The thickeners may be chosen from fatty acid amides (coconut diethanolamide or monoethanolamide, or oxyethylenated monoethanolamide of carboxylic acid alkyl ether), cellulose-based thickeners (hydroxyethylcellulose, hydroxypropylcellulose or carboxymethylcellulose), guar gum and its derivatives (hydroxypropylguar), gums of microbial origin (xanthan gum, scleroglucan gum), crosslinked homopolymers of acrylic acid or of acrylamidopropanesulfonic acid, and associative polymers (water-soluble polymers comprising hydrophilic zones and fatty-chain hydrophobic zones that are capable, in an aqueous medium, of reversibly associating with each other or with other molecules).

The compositions that are useful may moreover contain other types of surfactant such as anionic surfactants, nonionic surfactants, amphoteric or zwitterionic surfactants or cationic surfactants.

The compositions applied to the keratin fibers may also comprise one or more oxidation dye precursors: one or more oxidation bases and/or one or more couplers. By way of example, the oxidation bases are chosen from para-phenylenediamines, bis(phenyl)alkylenediamines, para-aminophenols, ortho-aminophenols and heterocyclic bases and the addition salts thereof.

The oxidation base(s) present is (are) generally present in an amount ranging from 0.001% to 20% by weight approximately, and preferably ranging from 0.005% to 6%, relative to the total weight of the dye composition.

The compositions may contain one or more couplers conventionally used for dyeing keratin fibers. Among these couplers, mention may be made especially of meta-phenylenediamines, meta-aminophenols, meta-diphenols, naphthalene-based couplers and heterocyclic couplers, and the addition salts thereof.

The coupler(s) is (are) generally present in an amount ranging from 0.001% to 20%, and preferably ranging from 0.005% to 6%, by weight approximately relative to the total weight of the dye composition.

In general, the addition salts of the oxidation bases and couplers that may be used in the context of the invention are chosen especially from the addition salts with an acid, such as the hydrochlorides, hydrobromides, sulfates, citrates, succinates, tartrates, lactates, tosylates, benzenesulfonates, phosphates and acetates, and the addition salts with a base, such as sodium hydroxide, potassium hydroxide, ammonia, amines or alkanolamines.

The compositions that are useful may also contain one or more additional direct dyes, which may especially be chosen from neutral, acidic or cationic nitrobenzene dyes, neutral, acidic or cationic azo direct dyes, neutral, acidic or cationic quinone and in particular anthraquinone direct dyes, azine direct dyes, triarylmethane direct dyes, indoamine direct dyes and natural direct dyes.

The direct dye(s) generally represent(s) from 0.001% to 20% by weight approximately, and even more preferably from 0.005% to 10% by weight approximately, relative to the total weight of the aqueous solution in which they are present.

The pH of the composition applied to the fibers is generally between 2 and 12 and preferably between 3 and 8. It may be adjusted to the desired value by means of acidifying or basifying agents usually used for dyeing keratin fibers, or alternatively using standard buffer systems.

Among the acidifying agents that may be mentioned, for example, are mineral or organic acids such as hydrochloric acid, orthophosphoric acid, sulfuric acid, carboxylic acids, for instance acetic acid, tartaric acid, citric acid or lactic acid, and sulfonic acids.

Among the basifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds of formula (II) below:

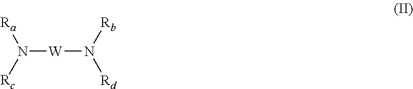

in which W is a propylene residue optionally substituted with a hydroxyl group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

When the composition comprises at least one oxidation dye precursor or when it is desired to perform lightening dyeing, an oxidizing agent may be used.

The oxidizing agents conventionally used for the oxidation dyeing of keratin fibers are, for example, hydrogen peroxide, urea peroxide, alkali metal bromates, persalts such as perborates and persulfates, peracids and oxidase enzymes, among which mention may be made of peroxidases, two-electron oxidoreductases such as uricases, and four-electron oxygenases, for instance laccases. Hydrogen peroxide is particularly preferred.

This oxidizing agent may also be present in any of the compositions that are useful in the invention or applied independently.

The oxidizing composition may also contain various adjuvants conventionally used in hair dye compositions and as defined previously.

The composition containing the colored polymer may be in various galenical forms, especially in the form of emulsions, microemulsions or nanoemulsions, or two-phase lotions, or in anhydrous form.

According to the process of the invention, the leave-on time of each of the compositions is not a limiting factor, since it is possible to obtain dyeing of keratin fibers instantaneously, which makes it possible to perform drying of the fibers immediately on completion of application of the composition comprising the colored polyelectrolyte. It is, however, possible to apply a leave-on time.

According to one particular embodiment, the process of the invention may comprise one or more pretreatment steps, for example a pretreatment step for a cationic polymer, this or these pretreatment step(s) possibly being followed by a rinsing step.

The process of the invention may be performed at a temperature ranging between room temperature (20-25° C.) and 200° C. and preferably between room temperature and 60° C.

According to one particular embodiment, the process of the invention is performed on dark hair, naturally or after dyeing, especially hair with a tone depth of less than 6 and preferably less than or equal to 4. Tone depths are commonly used in hair dyeing. They are especially described in “Sciences des traitements capillaires [Hair treatment sciences]” by C. Zviak, 1988, published by Masson, p. 278.

This classification is as follows:

### 1) Black

### 2) Brown

3) Dark chestnut

### 4) Chestnut

5) Light chestnut
6) Dark blond

### 7) Blond

8) Light blond
9) Very light blond
10) Light light blond

The examples that follow serve to illustrate the invention without, however, being limiting in nature.

### EXAMPLES

### Example 1

A two-phase lotion constituted of 80% water and 200 oleic acid and containing 0.5% of poly S-119 is applied to a lock of natural hair containing 90% white hairs. The application is not followed by a rinsing step or by a drying step. The orange coloration obtained is very attractive and fades out from the very first shampoo wash.

### Example 2

The lotion of Example 1 containing 0.3% Poly(Disperse Red 13 methacrylate) instead of the 0.5% of poly S-119 is applied to a lock of natural hair containing 90% white hairs. The application is immediately followed by a drying step with a hairdryer. The hair is dyed an attractive red color. This coloration fades out at the first shampoo wash.

### Example 3

A solution constituted of 3% alkyl (50/50 C₈/C₁₀) polyglucoside as an aqueous solution containing 60% water (qs 100) and containing 0.5% poly S-119 is applied using a foam-generating pump-dispenser bottle to a lock of natural hair containing 90% white hairs. The application is followed by a drying step. The hair is dyed orange.

### Example 4

A solution constituted of 3% alkyl (50/50 C₈/C₁₀) polyglucoside as an aqueous solution containing 60% water (qs 100) and containing 0.5% PAZO is applied using a foam-generating pump-dispenser bottle to a lock of natural hair containing 90% white hairs. The application is followed by a drying step. The hair is dyed yellow.

### Example 5

A solution constituted of hydroxyethylcellulose (2.5%), Fixate G-100 (4.2%) and containing 0.5% poly S-119 and 3-amino-2-methyl-1-propanol in an amount such that the solution has a pH of 7 is applied to a lock of natural grey hair containing 90% white hairs. The application is followed by a drying step. The orange coloration obtained is very attractive.

### Example 6

An aerosol is prepared from a composition comprising 33% DME, 20% ethanol, 0.5% poly S-119, 5% Luviset Si-PUR, 3-amino-2-methyl-1-propanol in an amount such that the pH of the composition is equal to 7, and water (qs 100%). The composition is then applied to a lock of natural grey hair containing 90% white hairs, by means of an aerosol. A very attractive orange coloration is obtained on application.

### Example 7

A lock of bleached hair (high alkaline solubility) is pretreated with an aqueous 10% 3-aminopropyl-triethylsilane solution at pH 8. After rinsing, an aqueous-alcoholic lotion (50/50) containing 0.5% PAZO is applied. The hair is dyed yellow. This coloration fades out at the first shampoo wash.

1. A process for dyeing the hair, comprising the application of a composition containing at least one anionic colored polymer comprising an anionic chromophore and at least one cosmetic active agent chosen from a non-silicone oil, an alkylpolyglucoside surfactant, a non-associative and nonionic, amphoteric, cationic or anionic carboxylic fixing polymer, and an organosilicon compound comprising less than 6 silicon atoms, this application not being followed by a step of rinsing the fibers.

2. The process as claimed in claim 1, in which the chromophore is substituted with at least one sulfonate, carboxylate, phosphate, phosphonate or sulfonate group.

3. The process as claimed in either of the preceding claims, in which the colored polymer is present in the composition in an amount of between 0.01% and 20% and preferably between 0.1% and 5%.

4. The process as claimed in any one of the preceding claims, in which the colored polymer(s) is (are) chosen from:

| | |
|---|---|
| Poly S-119 (orange) | 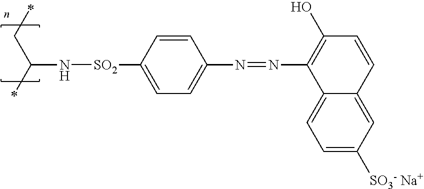 |
| Poly R-478 (violet) | 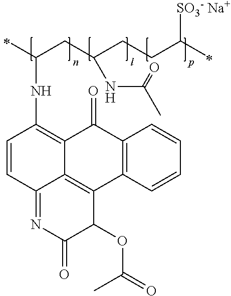 |
| PAZO (yellow) | 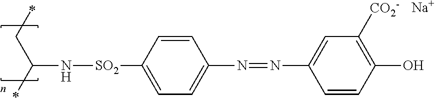 |
| Poly Black-863 (black) | 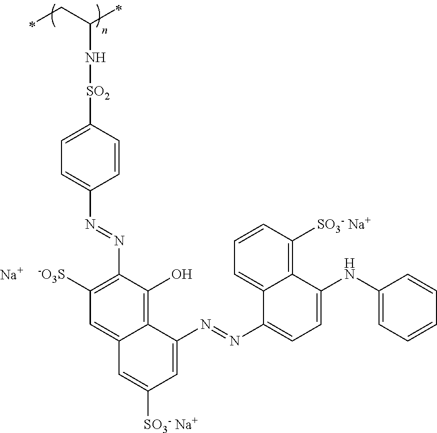 |
| Poly Y-606 (yellow) | 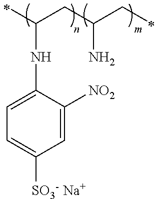 |
| Poly Y-636 (yellow) | 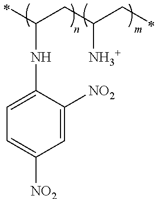 |
| Poly R-480 (red) | 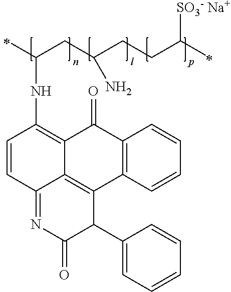 |
| Poly T-128 (yellow) | 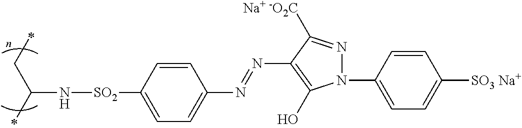 |
| Poly[methyl methacrylate- co- (fluorescein O-methacrylate)] (fluorescent yellow) | 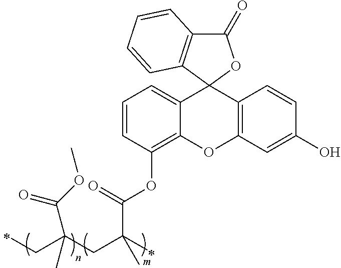 |
| Poly (Disperse Red 13 methacrylate) (red) | 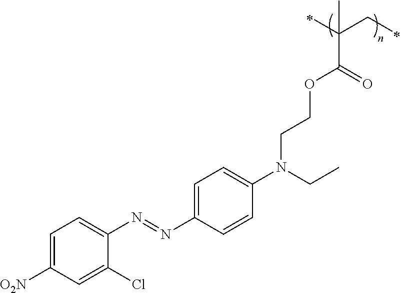 |

5. The process as claimed in any one of the preceding claims, in which the non-silicone oil is chosen from plant oils, mineral oils, synthetic oils, liquid fatty acid esters and liquid fatty acids.

6. The process as claimed in claim 5, in which the oil is chosen from liquid fatty acids, liquid fatty esters and plant oils.

7. The process as claimed in any one of the preceding claims, in which the amount of oil contained in the composition is between 0.01% and 95%, and preferably between 1% and 60%, by weight.

8. The process as claimed in any one of claims 1 to 7, in which the alkylpolyglucoside surfactant is represented by the following general formula:
R₁O—(R₂O)ₜ(G)ᵥ
in which R₁ represents a linear or branched alkyl and/or alkenyl radical containing from 8 to 24 carbon atoms approximately, an alkylphenyl radical in which the linear or branched alkyl radical contains from 8 to 24 carbon atoms, R₂ represents an alkylene radical containing from 2 to 4 carbon atoms approximately, G represents a sugar unit containing from 5 to 6 carbon atoms, t denotes a value ranging from 0 to 10 and preferably from 0 to 4, preferably 0 to 4, and v denotes a value ranging from 1 to 15.

9. The process as claimed in claim 8, in which the alkylpolyglucoside surfactants are such that R₁ denotes a saturated or unsaturated, linear or branched alkyl radical containing from 8 to 18 carbon atoms, t denotes a value ranging from 0 to 3, G may denote glucose, fructose or galactose, and v ranges from 1 to 15.

10. The process as claimed in claim 9, in which the alkylpolyglucoside surfactants are such that t is equal to 0 and G denotes glucose.

11. The process as claimed in any one of the preceding claims, in which the amount of alkylpolyglucoside surfactant is between 0.1% and 90% and preferably between 0.5% and 10% by weight relative to the total weight of the composition.

12. The process as claimed in any one of the preceding claims, in which the fixing polymer is chosen from anionic and nonionic polymers.

13. The process as claimed in claim 12, in which the fixing polymer is anionic.

14. The process as claimed in any one of the preceding claims, in which the fixing polymer is chosen from methyl vinyl ether/monoesterified maleic anhydride copolymers, acrylic acid/ethyl acrylate/N-tert-butyl acrylamide terpolymers, vinyl acetate/vinyl tert-butyl benzoate/crotonic acid terpolymers and crotonic acid/vinyl acetate/vinyl neododecanoate terpolymers, copolymers of methacrylic acid and of ethyl acrylate, vinylpyrrolidone/acrylic acid/lauryl methacrylate terpolymers, anionic copolymers of (meth)acrylic acids, of (meth)acrylic acid alkyl esters and of allyl methacrylate, vinylpyrrolidone homopolymers, and polyurethanes of the polyurethane/polysiloxane block anionic polymer type.

15. The process as claimed in claim 14, in which the amount of fixing polymer contained in the composition is between 0.01% and 20% and preferably between 0.5% and 10% by weight.

16. The process as claimed in any one of the preceding claims, in which the organosilicon compound is chosen from silicones and silanes.

17. The process as claimed in claim 16, in which the silane compound is chosen from 3-aminopropyl-triethoxysilane, 3-aminopropylmethyldiethoxysilane and 3-[(bis(hydroxyethyl)amino]propyltriethoxysilane.

18. The process as claimed in claim 8, in which the silane compound is 3-aminopropyltriethoxysilane.

19. The process as claimed in any one of the preceding claims, in which the organosilicon compound is included in an amount of between 0.01% and 95% and preferably between 1% and 60% by weight.

20. The process as claimed in any one of the preceding claims, in which the composition contains one or more organic and/or mineral salts.

21. The process as claimed in claim 20, in which the organic and/or mineral salts are chosen from sodium citrate, sodium chloride, ammonium sulfonate, magnesium chloride and calcium chloride.

22. The process as claimed in either of claims 20 and 21, in which the amount of salts is between 10⁻⁴ and 2 mol/l and preferably between 10⁻³ and 1 mol/l.

23. The process as claimed in any one of claims 1 to 22, for dyeing hair having a tone depth of less than or equal to 4.
